(19) United States
(12) Patent Application Publication
(10) Pub. No.: US 2024/0367161 A1
(43) Pub. Date: Nov. 7, 2024

(54) MULTIMODAL CHROMATOGRAPHY RESINS WITH ANIONIC AND CATIONIC LIGANDS

(71) Applicant: BIO-RAD LABORATORIES, INC., HERCULES, CA (US)

(72) Inventors: PEYTON RUSSELBURG, HERCULES, CA (US); BRIAN PANGANIBAN, HERCULES, CA (US); DANNI WANG, HERCULES, CA (US); JOSEPH POBLETE, HERCULES, CA (US); ALEXANDER POLGAR, HERCULES, CA (US)

(21) Appl. No.: 18/654,043

(22) Filed: May 3, 2024

Related U.S. Application Data
(60) Provisional application No. 63/464,263, filed on May 5, 2023.

(51) Int. Cl. B01J 43/00; B01D 15/38; B01J 47/02; C07K 1/22

(52) U.S. Cl. CPC B01J 43/00; B01D 15/3847; B01J 47/02; C07K 1/22

(57) ABSTRACT

The subject invention pertains to multimodal chromatography resins comprising anionic and cationic ligands on each resin particle and methods of synthesizing the multimodal chromatography resin by contacting a base with quaternary amines and/or tertiary amines on the surface of a substrate. The multimodal resins can be tuned by changing the ratios of cation to anions. The multimodal chromatography resins can be used for purifying proteins.

### CROSS-REFERENCE TO RELATED APPLICATION

This application claims the benefit of U.S. Provisional Application Ser. No. 63/464,263, filed May 5, 2023, the disclosure of which is hereby incorporated by reference in its entirety, including all figures, tables and amino acid or nucleic acid sequences.

### BACKGROUND OF THE INVENTION

The purification of proteins continues to present challenges. Proteins are often produced using engineered bacterial or eukaryotic cell lines to synthesize proteins of interest. The cell culture processes used for producing proteins are known to produce proteins with varying amounts of impurities. The purification of proteins can often include attempting to remove impurities including, for example, cells, cell debris, DNA, RNA, endotoxins, peptides, and virus and cell culture media components. Control over such impurities can impact numerous purified protein characteristics, including, for example, protein stability and efficacy.

Although various techniques are available for protein purification, the separation of proteins from impurities remains challenging. Therefore, there remains a need for compositions and methods for protein purification that possess a high affinity binding for the protein of interest with limited non-specific binding.

### BRIEF SUMMARY OF THE INVENTION

Provided herein is a chromatography media composed of a substrate (such as beads or other particulate substrates) that comprises two ligands, preferably one ligand is an anionic group and the second ligand is a cationic group. Also provided is a chromatography media (optionally contained within a column) comprising a substrate comprising anionic and cationic ligands (e.g., a plurality of beads or other substrate that comprises anionic ligands and cation ligands).

Also provided is a method of preparing the substrate comprising anionic and cationic ligands. In some embodiments, the method comprises incubating a substrate comprising quaternary amines, tertiary amines, or a combination thereof with base to form a substrate bearing a combination of cations and anions. In certain embodiments, the substrate comprising anionic and cationic ligands can be prepared by incubating with a base for a specific amount of time, at a specific temperature, and with a specific concentration of the base in order to achieve a specific ratio of cations to anions. In certain embodiments, the incubation temperature can be about 15° C. to about 150° C.: the concentration of the base can be about 0.1N to about 10N; and the incubation time can be about 1 second to about 3 months, FIG. 1 illustrates one embodiment of this process.

Also provided are methods of performing chromatography. In certain embodiments, the method comprises contacting a sample comprising a target molecule, such as, for example, a protein, with a substrate comprising anionic and cationic ligands as disclosed herein under conditions such that the target is captured by the substrate comprising anionic and cationic ligands; and collecting the target molecule in an eluate from the substrate comprising anionic and cationic ligands.

In some embodiments, the subject substrate comprising anionic and cationic ligands can limit the amount of contaminants or impurities that bind to the substrate. The ability of the substrate comprising anionic and cationic ligands to bind specifically to the target protein can be determined by the ratio of cations (e.g., quaternary amines and tertiary amines) to anions (e.g., carboxymethyl groups). In some embodiments, the protein is an antibody or another therapeutic protein.

### BRIEF DESCRIPTION OF THE DRAWINGS

FIG. 1 illustrates a proposed mechanism by altering a resin in which some amine ligands are removed and carboxylic acid groups replace some of the amine ligands.

FIG. 2A shows titration curves of chromatography resins with different distributions of Q and CM ligands. FIG. 2B shows the binding and elution profile of iodide on each of resins described in FIG. 2A.

FIG. 3 illustrates the conversion of a quaternary amine of the surface of the MP-HQ resin to a carboxymethyl group using NaOH.

FIG. 4 shows the performance testing of each resin treated with the NaOH, showing a trend of earlier elution times with an increased ratio of carboxymethyl (CM) group to quaternary amine.

FIG. 5 provides a comparison of the performance of a resin with 19% CM groups to a mixture of MP-HQ resins and Macro Prep-CM (MP-CM) resins with a measured ratio of 17% CM. The resin with the 19% CM, which has quaternary amines and CM ligands mixed on a single resin appears to retain better protein separation than the mixture of MP-HQ resins and MP-CM resins.

FIG. 6 shows that the binding affinity of the substrate comprising anionic and cationic ligands differs depending on if the quaternary amines and carboxymethyl group are distributed within a single resin (19% CM) or if the column contains a mixture of two resins (Q and CM mixed (17%)).

FIG. 7 shows scanning electron microscope (SEM) images of both MP-HQ resin (top row) and MP-HQ resin treated with sodium hydroxide for 48 hours (bottom row), which shows no significant difference in shape or structure.

FIG. 8 shows the different microenvironment for the substrate comprising quaternary amines and carboxymethyl groups distributed on a single resin and the mixture of two different substrates in which one contains a quaternary amine resin and the other possesses carboxymethyl resins.

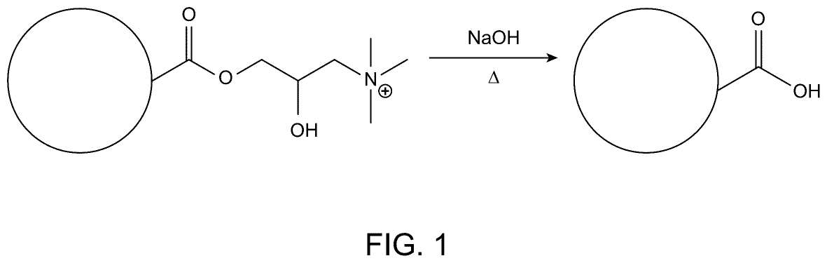

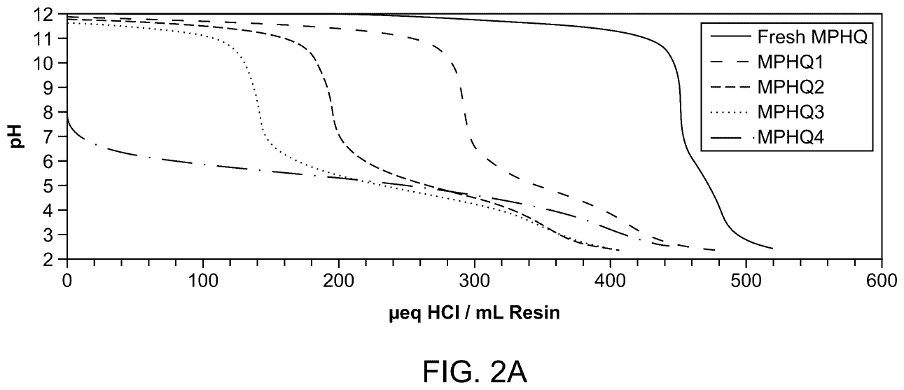

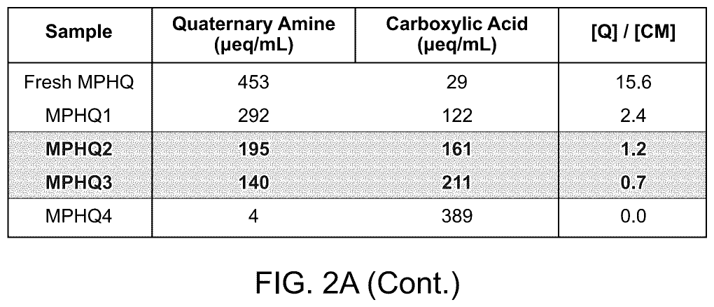

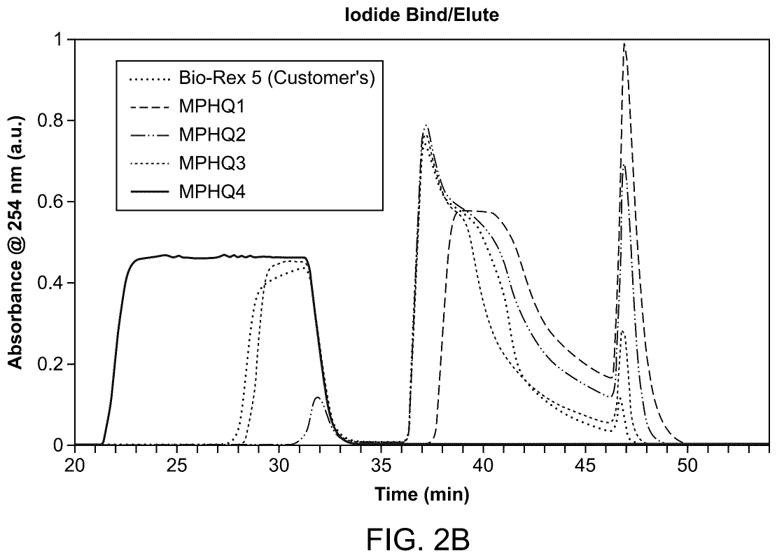

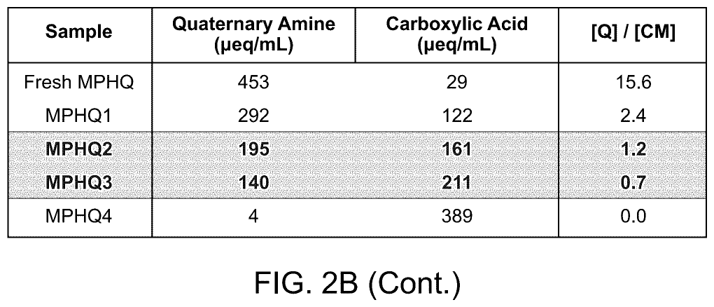

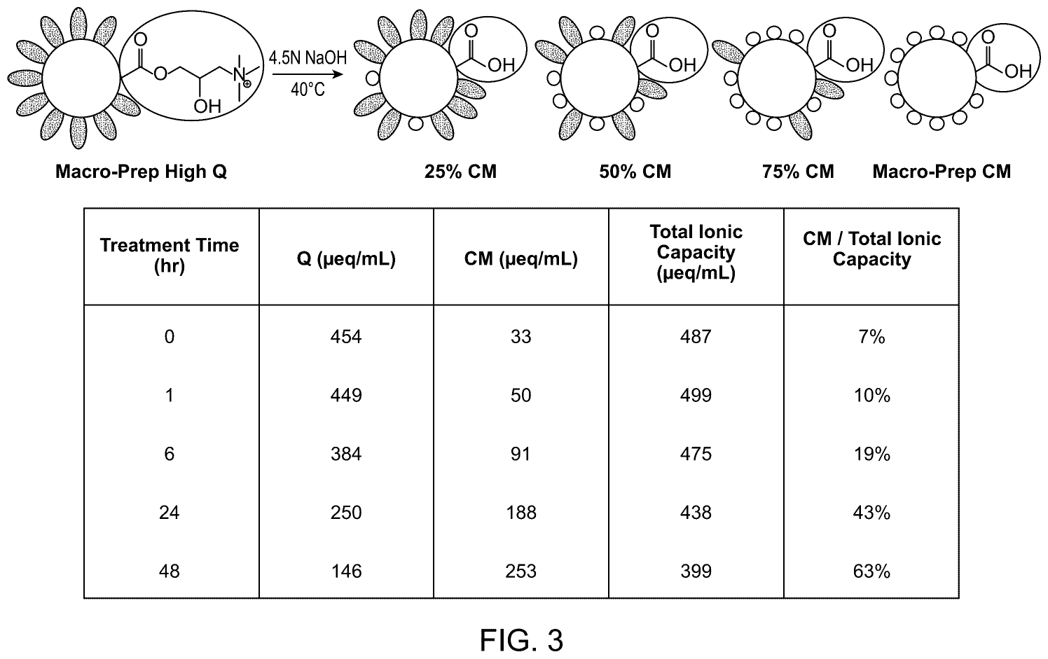

### DETAILED DESCRIPTION OF THE INVENTION

This disclosure relates to substrate comprising anionic and cationic ligands, methods of making substrate comprising anionic and cationic ligands, and methods of using substrate comprising anionic and cationic ligands.

### Definitions

As used herein, the singular forms “a”, “an” and “the” are intended to include the plural forms as well, unless the context clearly indicates otherwise. To the extent that the terms “including”, “Includes” “having”, “has” “with”, or variants thereof are used in either the detailed description and/or the claims, such terms are intended to be inclusive in a manner similar to the term “comprising” The transitional terms/phrases (and any grammatical variations thereof) “comprising”, “comprises”, “comprise”, “consisting essentially of”. “consists essentially of”, “consisting” and “consists” can be used interchangeably.

The phrase “consisting essentially of” or “consists essentially of” indicates that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of the lengths of polynucleotides where the terms “about” are used, these polynucleotides contain the stated number of bases or base-pairs with a variation of 0-10% around the value (X±10% b. In the context of compositions containing amounts of ingredients where the terms “about” or “approximately” are used, these compositions contain the stated amount of the ingredient with a variation (error range) of 0-10% around the stated value (X±10%), In the context of pH-, the term “about” or “approximately” is intended to include a value of ±0.2 unit of the stated pH.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The term “protein” is used to denote an amino acid polymer or a set of two or more interacting or bound amino acid polymers. The term applies to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers. The term “protein” includes therapeutic proteins, including, but are not limited to, enzymes, growth regulators, clotting factors, cytokines, antibodies, hormones, transcription factors and phosphoproteins.

The term “antibody” refers to an immunoglobulin or fragmentary form thereof. The term includes, but is not limited to, polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” encompasses composite forms including, but not limited to, fusion proteins containing an immunoglobulin moiety. “Antibody” also includes antibody fragments such as Fab, F(ab′)2, Fv, scFv, Fd, dAb, Fe and other compositions, whether or not they retain antigen-binding function.

The term “substrate comprising anionic and cationic ligands” as referred to within this disclosure is meant to denote a particulate substrate that comprises two ligands, one of which comprises an anionic group and the other of which comprises a cationic group. The phrase “substrate comprising anionic groups and cationic groups” can be used interchangeably with the phrase “substrate comprising anionic and cationic ligands” and are equivalent. Particulate substrates include spherical or oblong particles having diameters of between about 1 μm and about 1000 μm and can be monodisperse or polydisperse. For example, beads (also referred to as substrates can be identified as small-sized beads (<about 50 μm), medium-sized beads (about 50 to about 100 μm), or large-sized beads (greater than about 100 μm). Beads can, of course, have other shapes, such as a shard-like shape. Where spherical beads are used as a substrate, the spherical beads can be monodispersed or polydispersed in size distribution. In other embodiments, the substrate comprises structures selected from cubes, cuboids, prisms, pyramids, platonic solids, torus, cone, cylinder, spheres and mixtures thereof. In any of these embodiments, the beads have at least one dimension (length, width, diameter, and/or height) that is between about 1 μm and about 1000 μm; between about 5 μm and about 750 μm; between about 5 μm and about 600 μm; between about 5 μm and about 500 μm; between about 5 μm and about 400 μm; between about 5 μm and about 250 μm; between about 5 μm and about 150 μm; or between about 5 μm and about 100 μm.

The term “sample” refers to any composition containing a target molecule that is desired to be purified. The term “contaminant” or “impurity” refers to any substance that is to be removed from a sample. In some embodiments, the sample is a composition comprising contaminating substances from a cell culture.

The term “⁴ and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X. Y, and Z (i.e., any combination of X, Y, and Z).

The term “substrate” refers to a solid particulate substrate, such as beads, particles, or other particulate substrates. Such substrates comprise ligands, such as, for example, an anionic ligand and a cationic ligand (which may also be referred to as anionic groups and/or cationic groups herein). The substrate may be porous (micro-porous or macro-porous) or non-porous. The substrate may have quaternary amines, tertiary amines, sulfates, or a combination thereof on its surface. The substrate can be treated with a base to alter the ligand on the surface. Substrates can also comprise a linker coupled to the substrate. Particulate substrates can be formed from methacrylate, polystyrene, agarose, polyacrylamide, or any other suitable substrate. The substrate particles can have any shape, such a spherical or shard-like and can also be rigid or malleable. In certain embodiments, rigid substrates are preferred. The substrate can be polydisperse or monodisperse with respect to its size distribution.

### Producing Substrate Comprising Anionic and Cationic Ligands

In general terms, a chromatography substrate comprising anionic groups and cationic groups can be produced in the following generalized method. First, a desired chromatography support substrate possessing, for example, quaternary amines, tertiary amines, and/or sulfates, on the surface of the support substrate is chosen based on its chemical and mechanical properties. Thus, in some embodiments, the substrate contains only quaternary amines. In other embodiments, the substrate contains only tertiary amine groups. In other embodiments, the substrate contains only sulfate groups. In yet other embodiments, the substrate comprises a mixture of quaternary amine groups and tertiary amine groups. In yet other embodiments, the substrate comprises a mixture of quaternary amine groups and sulfate groups or a mixture of sulfate groups and tertiary amine groups. In various additional embodiments, any commercially available substrate comprising quaternary amines, tertiary amines, or a combination thereof can be used to make the substrate disclosed herein, such as, for example:
Macro-Prep® High Q Support (Bio-Rad Laboratories, Inc., Hercules, CA) with functional group —N⁺(CH₃)₃;
Macro-Prep® DEAE Resin (Bio-Rad Laboratories, Inc.) with functional group —N⁺(C₂H₅)₂;
AG® resins (Bio-Rad Laboratories, Inc.), including AG® 1 and AG® MP-1 with functional group —CH₂N⁺(CH₃)₃, AG® 2 with functional group —CH₂N⁺(CH₃)₂C₂H₄OH, AG® 3-X4 and AG® 4-X4 with functional group —CH₂N⁺(CH₃)₂, AG® 50W and AG® MP-50 with a strong sulfonic acid functional group, AG® 11 A8 with functional groups —CH₂N⁺(CH₃)₃ and —CH₂COO—, and AG® 501-X8 and AG® MP-501-X8(D), which have mixtures of AG® 50W and AG® 1 resins;
Bio-Rex Resins (Bio-Rad Laboratories, Inc.), including Bio-Rex 5 with functional group —N(CH₃)₂, Bio-Rex MSZ 1 Resin that is a mixture of AG® 50W and AG® 1 resins, and Bio-Rex 70 Resin with carboxylic acid exchange groups;
Nuvia™ Resins (Bio-Rad Laboratories, Inc.), including Nuvia™ HP-Q with functional group —N⁺(CH₃)₃, Nuvia™ HR-S with functional group —SO₃—, Nuvia™ S with functional group —SO₃—, Nuvia™ Q with functional group —N(CH₃)₃+, and Nuvia™ cPrime with a hydrophobic cation exchanger functional group;
UNOsphere™ Resins (Bio-Rad Laboratories, Inc.), including UNOsphere™ S Resin with functional group —SO₃— and UNOsphere™ Q functional group —N⁺(CH₃)₃; and
ENrich Q (Bio-Rad Laboratories, Inc.) with a quaternary ammonium functional group.

Following the selection of the desired chromatography support, the chromatography support substrate comprising the, for example, sulfates, quaternary amines, tertiary amines, or a combination thereof is treated with a base. The chromatography support substrate can then be washed to remove any residual base.

In certain embodiments, the substrate is a resin bead with a surface containing quaternary amines, sulfates, and/or tertiary amines, preferably the substrate has an ionically charged surface. Thus, in some embodiments, the substrate contains only quaternary amines. In other embodiments, the substrate contains only tertiary amine groups. In other embodiments, the substrate contains only sulfate amine groups. In yet other embodiments, the substrate comprises a mixture of quaternary amine groups and tertiary amine groups and/or sulfate groups. The resin beads can be made from various polymers can be used, including but not limited to polystyrene (e.g., polystyrene sulfonate), crosslinked polystyrene, divinylbenzene, cellulose, agarose, polymethacrylate, polyacrylamide, dextran, or any combination thereof. In certain embodiments, the quaternary amine can be, for example, hexadecyltrimethylammonium, chlorhexidine, quaternary ammonium methacrylate salt, or benzalkodium chloride. In certain embodiments, the tertiary amine can be, for example, benzyldimethylamine, pyridine, tertiary ammonium methacrylate salt, or imidazole.

In certain embodiments, the base is, for example, sodium hydroxide, lithium hydroxide, calcium hydroxide, strontium hydroxide, barium hydroxide, potassium hydroxide, cesium hydroxide, magnesium hydroxide, tetramethylammonium hydroxide, guanidine, rubidium hydroxide, or any combination thereof. In certain embodiment, the concentration of the base applied to the substrate containing the quaternary and/or tertiary amines is about 0.1N to about 10N, about 2.5N to about 7.5N, or about 4.5N. In certain embodiments, the substrate can be treated with the base for about 1 second to about 3 months, about 1 minute to about 10 weeks, about 2 minutes to about 6 weeks, about 5 minutes to about 4 weeks, about 10 minutes to about 2 weeks, about 15 minutes to about 1 week, about 30 minutes to about 3 days, about 1 hour to about 48 hours, or about 6 hours to about 24 hours at a temperature of about 15° C. to about 150° C., about 20° C. to about 125° C., about 25° C. to about 100° C., about 30° C. to about 75° C., about 35° C. to about 50° C., or about 40° C.

In certain embodiments, after the substrate is treated with a base, the substrate can be washed with, for example, deionized water. In certain embodiments, the deionized water can remove any free bases from the substrate. In certain embodiments, the substrate can be washed with the deionized water for about 1 second to about 24 hours months, about 1 minute to about 120 minutes, about 2 minutes to about 30 minutes, about 5 minutes to about 15 minutes, or about 10 minutes at a temperature of about 15° C. to about 150° C., about 20° C. to about 125° C., about 25° C. to about 100° C., about 30° C. to about 75° C., about 35° C. to about 50° C., or about 40° C.

Substrates of various diameters and/or dimensions can also be used, including small-sized particles or beads (<50 μm in at least one dimension e.g., length, width, diameter, and/or height), medium-sized particles or beads (about 50 to about 100 μm in at least one dimension e.g., length, width, diameter, and/or height), and large-sized particles or beads (greater than about 100 μm in at least one dimension e.g., length, width, diameter, and/or height).

In certain embodiments, the resulting substrates comprise a cationic group and an anionic group and can have a ratio of cationic groups (e.g., sulfates, quaternary and/or tertiary amine) to anionic groups (e.g., carboxymethyl groups) of less than or equal to about 50:1, about 25:1, about 15.5:1, about 2.4:1, about 1.2:1, about 1:1, or about 0.7:1. Other embodiments provide for a ratio of cationic groups to anionic groups that is represented by X:1, where X is any value between 0.2 and 50, the value being incrementally increased by 0.1 or 0.05. Yet other embodiments provide for a ratio of cationic groups to anionic groups that is represented by X:1, where X is any value between 0.2 and 50, the value being incrementally increased by 0.1 or 0.05. Yet other embodiments provide for a ratio of cationic groups to anionic groups that ranges from 0.5:1 to 50:1.

In certain embodiments, the amount of anionic groups attached to the substrate relative to the total ionic capacity of the substrate can be about 5% to about 95%, about 5% to about 65%, about 7% to about 63%, about 10% to about 43%, or about 19%. The amount of cationic groups attached to the substrate, relative to the amount of anionic groups is such that the total amount of anionic groups and cationic groups on the surface is 100%. Thus, the amount of cation attached to the surface relative to the total ionic capacity of the substrate can be about 5% to about 95%, about 35% to about 95%, about 37% to about 93%, about 67% to about 90%, or about 81%. As will be appreciated, the total ionic capacity is all (100%) of the anionic groups and cationic groups present on said substrate.

In other embodiments, the amount of anionic groups attached to the surface of the substrate is about X %, where X is any integer between 1 and 99 and the amount of cationic groups attached to the surface of the substrate is about Y %, where Y is any integer between 1 and 99, the sum total of the selected X and Y values being 100 (100%).

In certain embodiments, the total ionic capacity can be about 100 μeq/mL to about 1000 μeq/mL, about 250 μeq/mL to about 750 μeq/mL, about 350 μeq/mL to about 500 μeq/mL, about 399 μeq/mL to about 499 μeq/mL, about 438 μeq/mL to about 487 μeq/mL, or about 475 μeq/mL. In certain embodiments, the cationic capacity can be about 10 μeq/mL to about 1000 μeq/mL, about 50 μeq/mL to about 750 μeq/mL, about 100 μeq/mL to about 500 μeq/mL, about 146 μeq/mL to about 454 μeq/mL, about 250 μeq/mL to about 449 μeq/mL, or about 384 μeq/mL. In certain embodiments, the anionic capacity can be about 1 μeq/mL to about 1000 μeq/mL, about 10 μeq/mL to about 750 μeq/mL, about 15 μeq/mL to about 500 μeq/mL, about 33 μeq/mL to about 253 μeq/mL, about 50 μeq/mL to about 188 μeq/mL, or about 91 μeq/mL.

Substrates comprising anionic groups (ligands) and cationic groups (ligands) can be produced by contacting a solid particulate substrate comprising a quaternary amine, sulfate, and/or a tertiary amine with a base. Methods of producing a substrate comprising anionic and cationic ligands comprise, for example:
a) contacting a solid substrate comprising quaternary amines, sulfates, and/or tertiary amines with a base (for example, 4.5N sodium hydroxide at 40° C. for about 1 second to about 48 hours); and
b) optionally, washing the solid substrate contacted with the base (e.g., sodium hydroxide solution with sodium chloride at about 18° C. to about 25° C. for about 1 second to about 1 hour);
c) optionally, washing the solid substrate contacted with the sodium hydroxide solution with deionized water at about 18° C. to about 25° C. for about 1 second to about 1 hour.

### Methods of Use

Protein purification utilizing a substrate comprising anionic and cationic ligands can be achieved by conventional means known to those of skill in the art. Examples of proteins include but are not limited to antibodies, enzymes, growth regulators, clotting factors, transcription factors, and phosphoproteins. In many such conventional procedures, the substrate comprising anionic and cationic ligands, prior to use, is equilibrated with a buffer (“an equilibration buffer”) at the pH that will be used for the binding of the target molecule (e.g., antibody or non-antibody protein). Equilibration can be done with respect to all features that will affect the binding environment, including ionic strength and conductivity when appropriate.

In some embodiments, the substrate comprising anionic and cationic ligands described herein can be used in “bind-elute” mode to purify a target molecule or two or more target molecules from a sample. In some embodiments, following binding of the target molecule to the subject substrate comprising anionic and cationic ligands, a change in the pH and or salt content of the elution buffer can be used to elute the target molecule.

In some embodiments, once the substrate comprising anionic and cationic ligands is equilibrated, the sample containing a target molecule is loaded onto the substrate comprising anionic and cationic ligands (a “loading step”). The sample can optionally be diluted or equilibrated into the equilibration buffer and the target molecule can be allowed to bind to the substrate comprising anionic and cationic ligands. Once bound to the substrate comprising anionic and cationic ligands, the substrate comprising anionic and cationic ligands can be washed with a “wash solution” to remove contaminants not bound to the substrate comprising anionic and cationic ligands.

Non-limiting examples of solutions suitable for use in connection with chromatography (e.g., wash solution, equilibration solution) using the disclosed substrate comprising anionic and cationic ligands include water (e.g., deionized water) or a buffer, such as, for example Bis-Tris at a pH of about 7. Other non-limiting examples of buffers suitable for use include phosphate buffers (e.g., monosodium, disodium, and/or trisodium phosphate buffers), ammonium phosphate, alkali calcium phosphate, and potassium phosphate (monobasic and/or dibasic) buffers. Yet other buffers include acetate buffers (e.g., sodium acetate), acetic acid, malonic acid, succinate buffers, imidazole buffers, arginine buffers, glycine buffers, HEPES (2-[4-(2-hydroxyethyl)piperazin-1-yl]ethanesulfonic acid), BICINE (N,N-Bis(2-hydroxyethyl)glycine), Bis-tris (Bis-tris methane), Bis-tris propane, TRIS (Tris(hydroxymethyl)aminomethane), MES (2-Morpholinoethanesulfonic acid monohydrate or ACES (N-(2-Acetamido)-2-aminoethanesulfonic acid) buffers. The buffers are, generally, prepared at concentrations of about 10 mM to about 100 mM, or about 20-50 mM. These buffers, may, optionally, further comprise a salt. Examples of salts that can be used for this purpose are alkali metal and alkaline earth metal halides, notably sodium and potassium halides, and as a specific example NaCl or KCl. Other salts include sulfates, acetates, bromides, perchlorates, iodides, thiocyanates and suitable cations, such as ammonium, alkali metals and alkaline earth metals.

In some embodiments, the substrate comprising anionic and cationic ligands may be washed with a wash buffer, such as the equilibration buffer, to remove any unbound proteins or substances that may have been present in the source liquid. The bound protein (e.g., antibody or non-antibody protein, as desired) can be subsequently eluted with an elution buffer. Isocratic elution, stepwise elution in which buffer conditions or salt conditions are changed, or gradient elution using, for example, a buffer at a constant pH and a salt gradient or a gradient of phosphate ions can be used for eluting a protein of interest.

In other embodiments, the binding and washing steps are performed with the inclusion of at least one salt in the sample and wash liquids. Examples of salts that can be used for this purpose are alkali metal and alkaline earth metal halides, notably sodium and potassium halides, and as a specific example sodium chloride. The concentration of the salt can vary; in most cases, an appropriate concentration will be one within the range of about 10 mM to about 2 M, about 10 mM to about 1.5 M, about 10 mM to about 1 M, about 10 mM to about 750 mM, about 10 mM to about 500 mM, about 10 mM to about 250 mM, about 20 mM to about 150 mM, about 20 mM, or about 150 mM. In some instances, bound proteins can be eluted with a salt gradient. In other embodiments, a stepwise elution can be utilized in which the amount of salt contained within a buffer is altered (e.g., increased) and passed over the column to elute bound protein. In some embodiments, the concentration to salt ranges: between X mM and 1000 mM, where X is any integer between 1 and 999; between X mM and 750 mM, where X is any integer between 1 and 749; between X mM and 500 mM, where X is any integer between 1 and 499; between X mM and 250 mM, where X is any integer between 1 and 249; between X mM and 100 mM, where X is any integer between 1 and 99; between X mM and 50 mM, where X is any integer between 1 and 49.

The substrate comprising anionic and cationic ligands can be utilized in any conventional configuration, including packed columns and fluidized or expanded-bed columns, and by any conventional method, including batchwise modes for loading, washes, and elution, as well as continuous or flow-through modes. The use of a packed flow-through column is particularly convenient, both for preparative-scale extractions and analytical-scale extractions. A column may thus range in diameter from about 1 mm to about 1 m, and in height from about 1 cm to about 30 cm or more.

The chromatographic steps described herein can be performed in a conventional purification configuration including, but not limited to, packed columns and fluidized or expanded-bed columns and by any conventional chromatography method including batch modes for loading, washing, and elution, as well as continuous or flow-through modes. In some embodiments, the medium is packed in a column having a diameter ranging from less than 0.5 centimeter to more than a meter and a column height ranging from less than one centimeter to more than 30 centimeters. In other embodiments, the substrate comprising anionic and cationic ligands is provided in a spin column.

In other embodiments, the substrate comprising anionic and cationic ligands is provided in a chromatography column, the sample is applied to the top of the column and gravity forces the sample, wash buffers and/or elution buffers through the column. In other embodiments, a column containing the substrate comprising anionic and cationic ligands can be run with or without pressure and from top to bottom or bottom to top, and the direction of the flow of fluid in the column can be reversed during the process. In some cases, it can be advantageous to reverse the flow of liquid while maintaining the packed configuration of the packed bed. The methods described herein can be used for purifying many types of target molecules, including naturally occurring proteins, antibodies, and recombinant proteins.

The output from a chromatography column comprising a substrate comprising anionic and cationic ligands can be monitored for the presence of the target molecule or other components of the sample, as desired, to determine fractions that contain the target molecule and that are free, or at least have a reduced amount, of contaminant compared to the original sample. In some embodiments, at least 90%, 95%, 99% of the contaminant in the sample is removed in the resulting purified target molecule fractions. An exemplary method for measuring output includes monitoring a characteristic absorbance wavelength (e.g., 254 nm) for the target molecule. The term “fraction” is used to refer to a portion of the output of chromatography and is not intended to limit how the output is collected or whether the output is collected in parts or continuously.

Any antibody preparation can be used in the present invention, including unpurified or partially purified antibodies from natural, synthetic, and/or recombinant sources. Unpurified antibody preparations can come from various sources such as, for example, plasma, serum, ascites, milk, plant extracts, bacterial lysates, yeast lysates, or conditioned cell culture media. Partially purified preparations can come from unpurified preparations that have been processed by at least one chromatography, precipitation, other fractionation step, or any combination thereof. In some embodiments, the antibodies have not been purified by protein affinity prior to purification.

In certain embodiments, the substrate comprising anionic and cationic ligands can be used for purification of non-antibody proteins, including therapeutic proteins. Examples of therapeutic proteins include, but are not limited to, enzymes, growth regulators, clotting factors, transcription factors and phosphoproteins.

In certain embodiments, the amount of the anions and cations and the surface of substrate comprising anionic and cationic ligands can be altered based on the properties of the protein that is being purified. In certain embodiments, the reaction of the base with the quaternary and/or tertiary amines does not alter the general shape or underlying structure of the substrate.

All patents, patent applications, provisional applications, and publications referred to or cited herein are incorporated by reference in their entirety, including all figures and tables, to the extent they are not inconsistent with the explicit teachings of this specification.

### Additional Disclosure and Claimable Subject Matter

1. A substrate comprising anionic and cationic ligands made by a method comprising:
a) contacting a solution comprising a base with a substrate comprising a sulfate, quaternary amine, and/or a tertiary amine on a surface of the substrate.

2. The substrate comprising anionic and cationic ligands of embodiment 1, the method further comprising:
b) washing the substrate contacted with base with a wash solution.

3. The substrate comprising anionic and cationic ligands of embodiment 1, wherein the substrate comprises methacrylate, polystyrene, agarose, divinylbenzene, polyacrylamide, cellulose, dextran, or mixtures thereof.

4. The substrate comprising anionic and cationic ligands of any one of embodiments 1-3, wherein the base is sodium hydroxide, lithium hydroxide, calcium hydroxide, strontium hydroxide, barium hydroxide, potassium hydroxide, cesium hydroxide, magnesium hydroxide, tetramethylammonium hydroxide, guanidine, rubidium hydroxide, or any combination thereof.

5. The substrate comprising anionic and cationic ligands of any one of embodiments 1-4, wherein the base is at a concentration of about 0.1N to about 10N.

6. The substrate comprising anionic and cationic ligands of embodiment 5, wherein the base is at a concentration of about 4.5N.

7. The substrate comprising anionic and cationic ligands of any one of embodiments 1-6, wherein step a) occurs at a temperature of about 15° C. to about 150° C.

8. The substrate comprising anionic and cationic ligands of embodiment 7, wherein step a) occurs at a temperature of 40° C.

9. The substrate comprising anionic and cationic ligands of any one of embodiments 1-8, wherein step a) occurs for about 1 second to about 3 months.

10. The substrate comprising anionic and cationic ligands of embodiment 9, wherein step a) occurs for about 1 second to about 48 hours.

11. The substrate comprising anionic and cationic ligands of any preceding embodiment, wherein the substrate comprises cubes, cuboids, prisms, pyramids, platonic solids, torus, cone, cylinder, spheres and mixtures thereof having at least one dimension that is between about 1 μm and about 1000 μm; between about 5 μm and about 750 μm; between about 5 μm and about 600 μm; between about 5 μm and about 500 μm; between about 5 μm and about 400 μm; between about 5 μm and about 250 μm; between about 5 μm and about 150 μm; or between about 5 μm and about 100 μm as a length, width, diameter, and/or height.

12. The substrate comprising anionic and cationic ligands of embodiment 11, wherein the substrate is a sphere having a diameter of: between about 1 μm and about 1000 μm; less than about 50 μm, between about 50 μm and about 100 μm; or greater than 100 μm and less than about 1000 μm.

13. The substrate comprising anionic and cationic ligands of any one of embodiments 1-12, wherein a portion of the sulfate, quaternary amine and/or the tertiary amine on the substrate is converted to an anionic group by treating the substrate with the solution comprising a base.

14. The substrate comprising anionic and cationic ligands of embodiment 13, wherein the anionic group is a carboxymethyl group.

15. The substrate comprising anionic and cationic ligands of embodiment 13 or 14, wherein the substrate comprising anionic and cationic ligands has a ratio of cationic groups to anionic groups that is less than or equal to about 50:1, about 25:1, about 15.5:1, about 2.4:1, about 1.2:1, about 1:1, about 0.7:1, about 0.5:1, about 0.3:1, or about 0.2:1.

16. The substrate comprising anionic and cationic ligands of embodiment 13 or 14, wherein the substrate comprising anionic and cationic ligands has:
a) an amount of anionic groups (ligands) on the surface, relative to the total ionic capacity of the substrate, that are about 5% to about to about 95%, about 7% to about 63%, about 10% to about 43%, or about 19% of the total ionic capacity of the substrate, the total ionic capacity being all (100%) of the anionic groups and cationic groups present on said substrate comprising anionic groups and cationic groups; or
b) the amount of anionic groups (ligands) attached to the surface of the substrate is about X %, where X is any integer between 1 and 99 and the amount of cationic groups (ligands) attached to the surface of the substrate is about Y %, where Y is any integer between 1 and 99, the sum total of the selected X and Y values being 100 (100%).

17. A chromatography column comprising a plurality of substrate comprising anionic and cationic ligands according to embodiments 1-16.

18. A method of performing chromatography, the method comprising:
contacting a sample comprising a target molecule to a plurality of substrates comprising anionic and cationic ligands according to any one of embodiments 1-16 or a chromatography column according to embodiment 17 under conditions such that the target molecule is captured by the plurality of substrate comprising anionic and cationic ligands;
optionally washing the plurality of substrate comprising anionic and cationic ligands with a solution; and
eluting and collecting the target molecule.

19. The method of embodiment 18, wherein the target molecule is a protein.

20. The method of embodiment 18, wherein the solution comprises Bis-Tris.

21. A method of making a substrate comprising anionic and cationic ligands comprising:
a) contacting a solution comprising a base with a substrate comprising a sulfate, quaternary amine, and/or a tertiary amine on a surface of the substrate.

22. The method of embodiment 21, the method further comprising washing the substrate contacted with base with a wash solution and, optionally, recovering the substrate.

23. The method of embodiment 21, wherein the substrate comprises methacrylate, polystyrene, agarose, divinylbenzene, polyacrylamide, cellulose, dextran, or mixtures thereof.

24. The method of any one of embodiments 21-23, wherein the base is sodium hydroxide, lithium hydroxide, calcium hydroxide, strontium hydroxide, barium hydroxide, potassium hydroxide, cesium hydroxide, magnesium hydroxide, tetramethylammonium hydroxide, guanidine, rubidium hydroxide, or any combination thereof.

25. The method of any one of embodiments 21-24, wherein the base is at a concentration of about 0.1N to about 10N.

26. The method of embodiment 25, wherein the base is at a concentration of about 4.5N.

27. The method of any one of embodiments 21-26, wherein step a) occurs at a temperature of about 15° C. to about 150° C.

28. The method of embodiment 27, wherein step a) occurs at a temperature of 40° C.

29. The method of any one of embodiments 21-28, wherein step a) occurs for about 1 second to about 3 months.

30. The method of embodiment 29, wherein step a) occurs for about 1 second to about 48 hours.

31. The method of any preceding embodiment, wherein the substrate comprises cubes, cuboids, prisms, pyramids, platonic solids, torus, cone, cylinder, spheres and mixtures thereof having at least one dimension that is between about 1 μm and about 1000 μm; between about 5 μm and about 750 μm; between about 5 μm and about 600 μm; between about 5 μm and about 500 μm; between about 5 μm and about 400 μm; between about 5 μm and about 250 μm; between about 5 μm and about 150 μm; or between about 5 μm and about 100 μm as a length, width, diameter, and/or height.

32. The method of embodiment 31, wherein the substrate is a sphere having a diameter of: between about 1 μm and about 1000 μm; less than about 50 μm, between about 50 μm and about 100 μm; or greater than 100 μm and less than about 1000 μm.

33. The method of any one of embodiments 21-32, wherein a portion of the sulfate, quaternary amine and/or the tertiary amine on the substrate is converted to an anionic group by treating the substrate with the solution comprising a base.

34. The method of embodiment 33, wherein the anionic group is a carboxymethyl group.

35. The method of embodiment 33 or 34, wherein the substrate comprising anionic and cationic ligands has a ratio of cationic groups to anionic groups that is less than or equal to about 50:1, about 25:1, about 15.5:1, about 2.4:1, about 1.2:1, about 1:1, about 0.7:1, about 0.5:1, about 0.3:1, or about 0.2:1.

36. The method of embodiment 33 or 34, wherein the substrate comprising anionic and cationic ligands has:
a) an amount of anionic groups (ligands) on the surface, relative to the total ionic capacity of the substrate, that are about 5% to about to about 95%, about 7% to about 63%, about 10% to about 43%, or about 19% of the total ionic capacity of the substrate, the total ionic capacity being all (100%) of the anionic groups and cationic groups present on said substrate comprising anionic groups and cationic groups; or
b) the amount of anionic groups (ligands) attached to the surface of the substrate is about X %, where X is any integer between 1 and 99 and the amount of cationic groups (ligands) attached to the surface of the substrate is about Y %, where Y is any integer between 1 and 99, the sum total of the selected X and Y values being 100 (100%).

### EXAMPLE 1—Synthesizing a Resin with Cationic and Anionic Ligand

Bio-Rad Laboratories' Macro-Prep High Q was chosen as a strong anion exchange resin substrate. The Macro-Prep High Q resin was placed into a bottle with 4.5N sodium hydroxide. This bottle was heated to 40° C. and agitated for various incubation times, accomplished with a shaking water bath, belt heater and orbital shaker, or reactor, etc. After treatment, the NaOH was removed and the resin rinsed with water. The polymer resin was loaded into a column and set up to a liquid chromatography system. Flow rate was adjusted to change the contact time from 1 second to 48 hours and a solution comprising 4.5N sodium hydroxide flowed through the column at an ambient temperature of 40° C. After the sodium hydroxide flowed through the column, deionized water flowed through the column at about 18° C. to about 25° C. for about 1 second to about 1 hour.

### EXAMPLE 2—Purifying a Protein

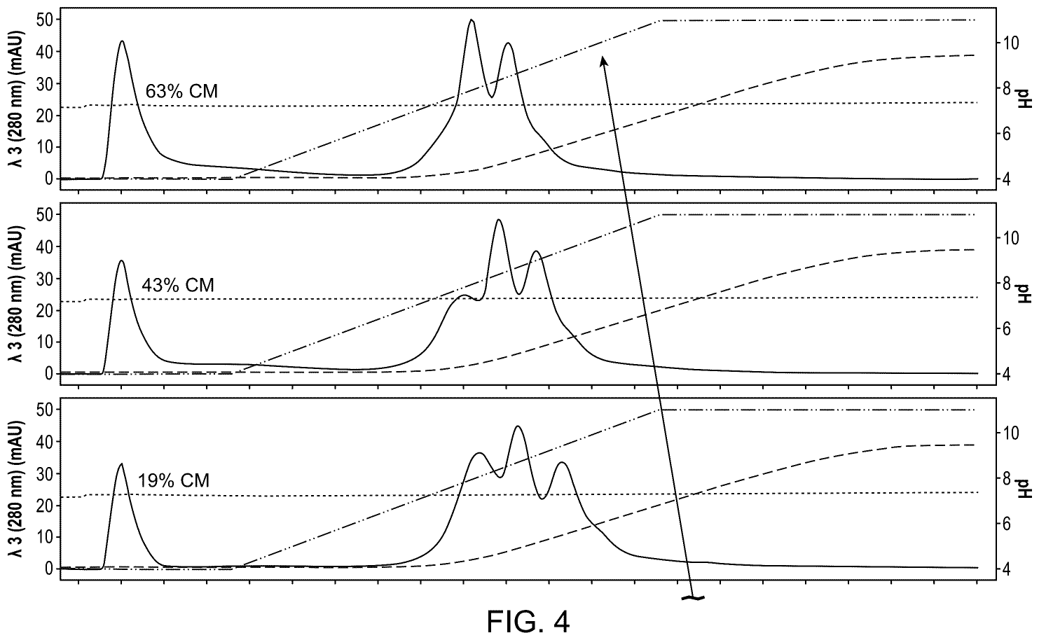

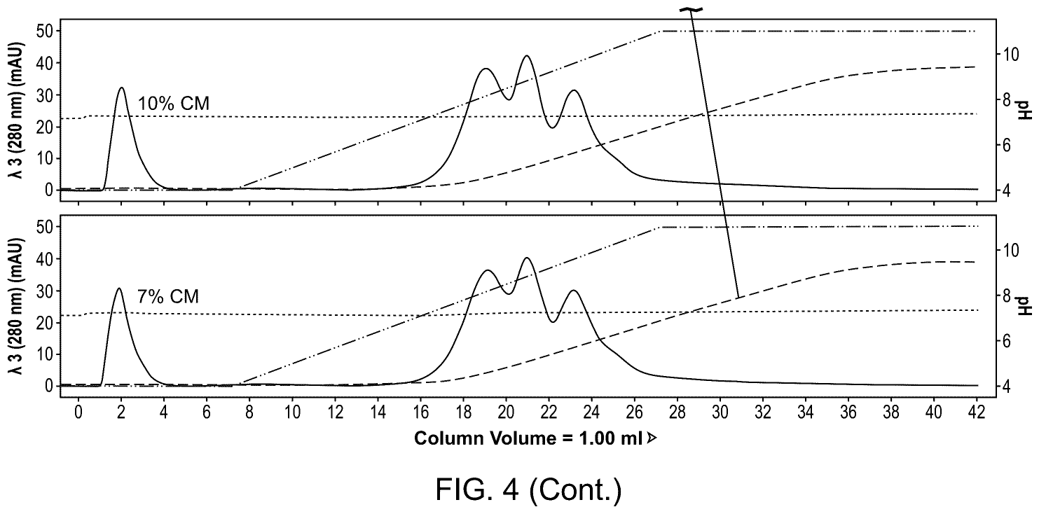

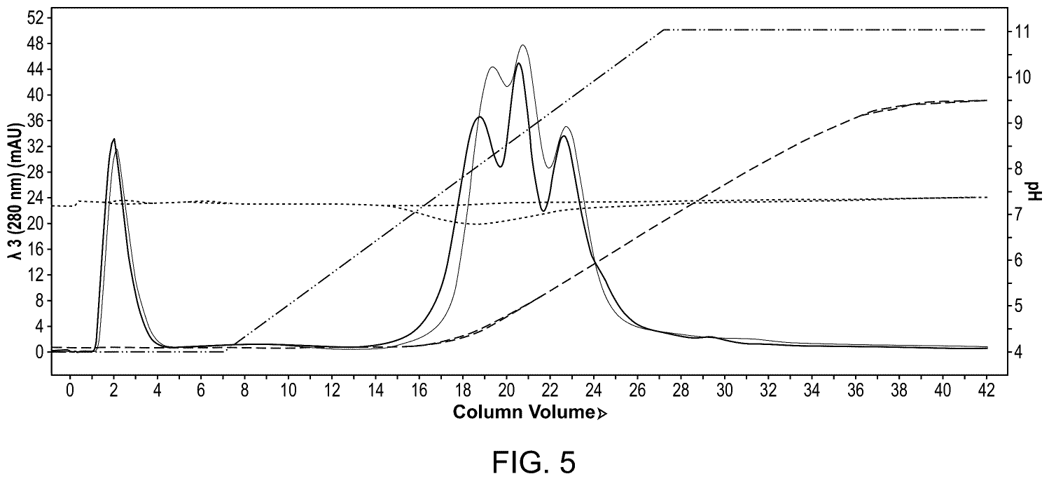

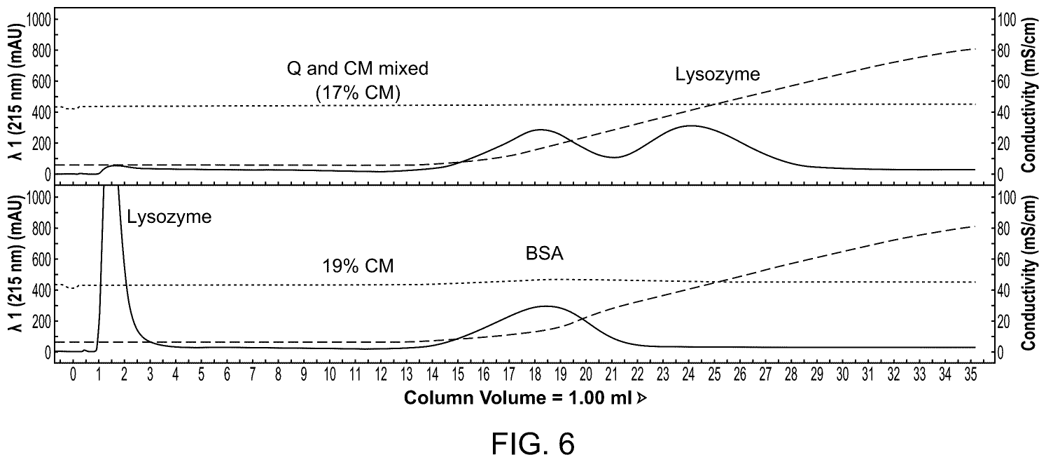

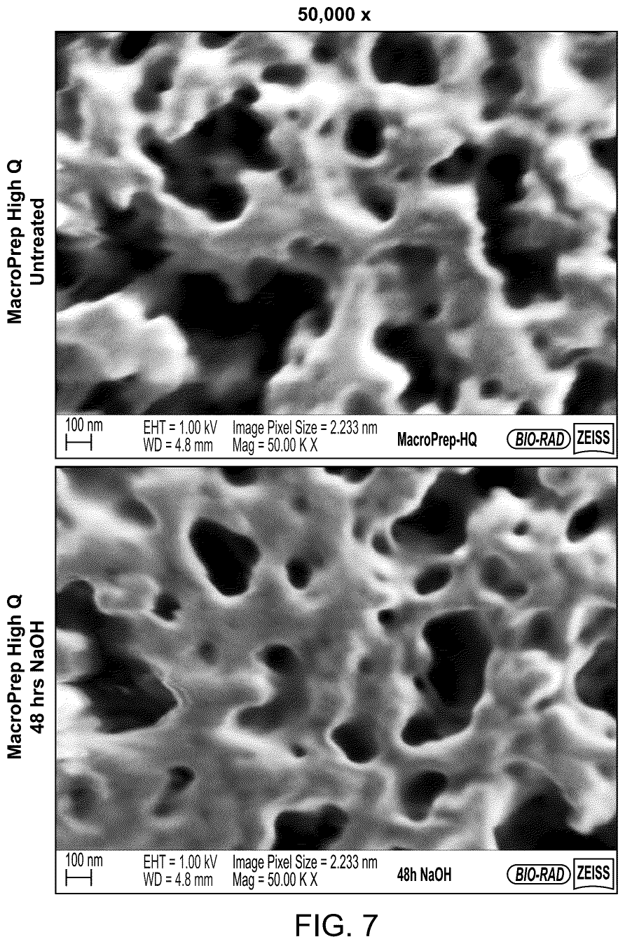

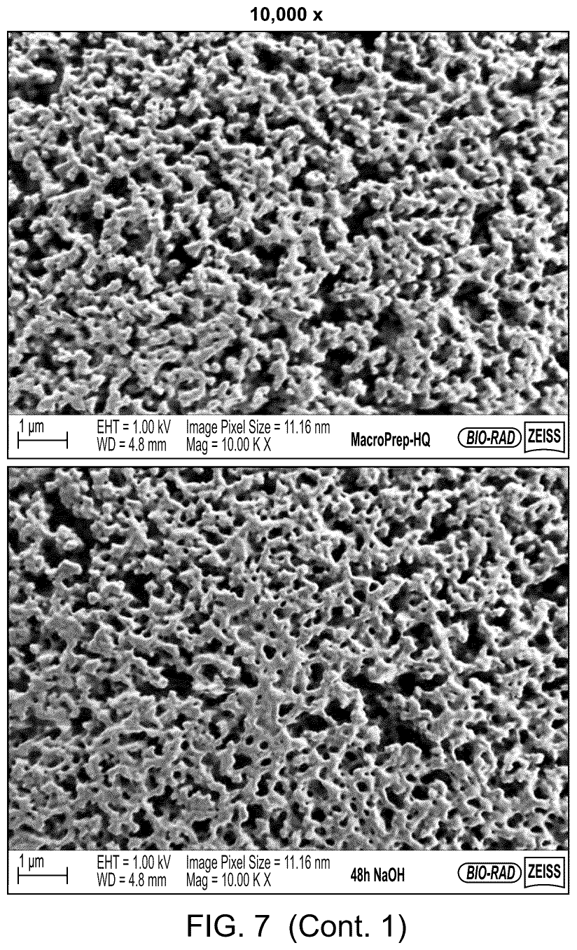

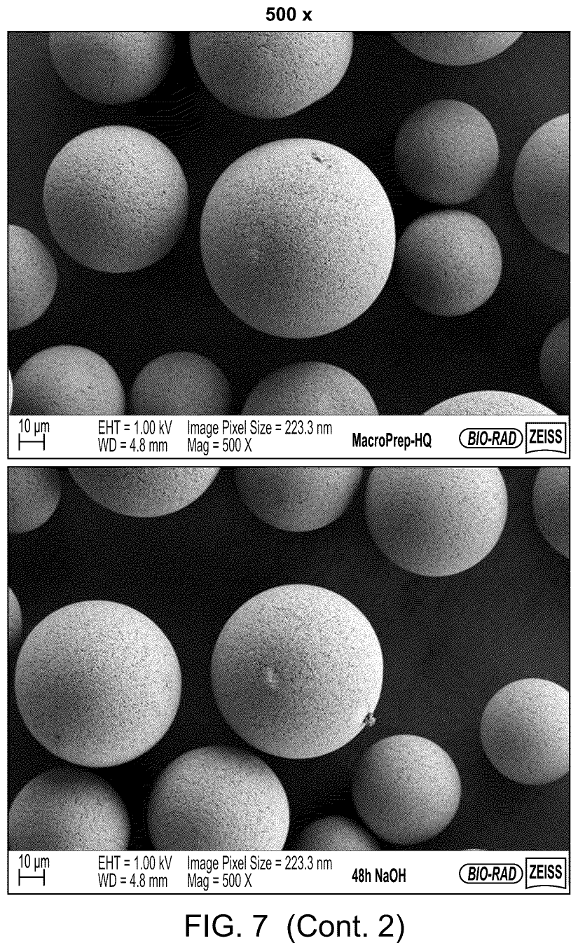

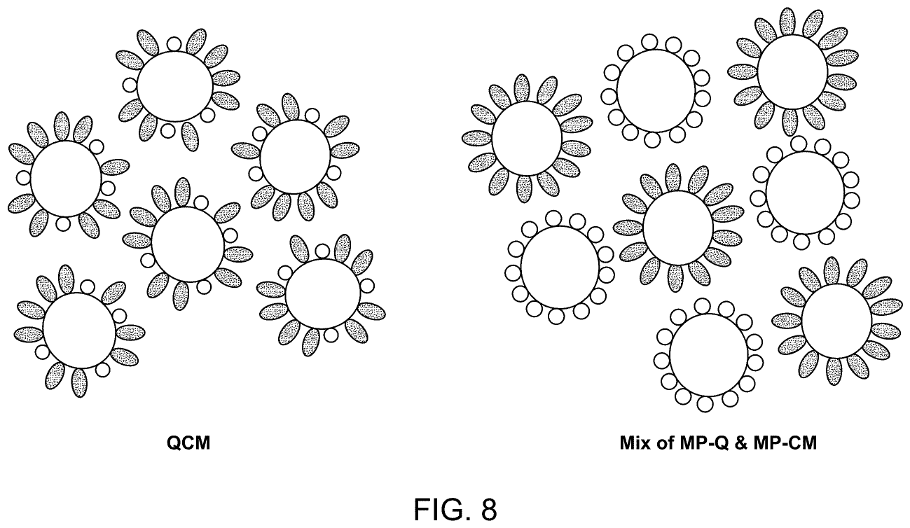

This example demonstrates the chromatographic performance of Macro-Prep HQ treated with sodium hydroxide at 40° C. for various periods of time (material from Example 1). Macro-Prep HQ treated with sodium hydroxide is packed in a chromatography column. The conditions were as followed:
Column: 1 mL (0.56×1.0 cm)
Flow Rate: 2 mL/min
Sample: Lysozyme (FIG. 6)
   Bovine serum albumin (BSA) (FIG. 6)
   1 mL anion exchange standard (myoglobin (pI ˜7.2), conalbumin (pI ˜6.85), ovalbumin (pI ˜5.19), and soybean trypsin inhibitor (pI ˜4.95) resuspended in 50 mM Bis-Tris pH 7.0, 20 mL) (FIGS. 4 and 5)
Equilibration: Buffer A: 50 mM Bis-Tris, pH 7.0
   Buffer B: 50 mM Bis-Tris, 1 M Sodium Chloride, pH 7.0
   (Step 1) 100% B, 15 Column Volumes;
   (Step 2) 100% A, 45 Column Volumes;
Post-Loading: Buffer B: 50 mM Bis-Tris, 1 M Sodium Chloride, pH 7.0
   (Step 1) 100% A, 5 Column Volumes;
Elution: Buffer A: 50 mM Bis-Tris, pH 7.0
   Buffer B: 50 mM Bis-Tris, 1 M Sodium Chloride, pH 7.0
   (Step 1) 0-100% B, 20 Column Volumes;
   (Step 2) 100% B, 10 Column Volumes;
Regeneration: Buffer B: 50 mM Bis-Tris, 1 M Sodium Chloride, pH 7.0
   (Step 1) 100% B, 5 Column Volumes.

We claim:

1. A substrate comprising anionic and cationic ligands made by a method comprising:
a) contacting a solution comprising a base with a substrate comprising a sulfate, quaternary amine, and/or a tertiary amine on a surface of the substrate.

2. The substrate comprising anionic and cationic ligands of claim 1, the method further comprising:
b) washing the substrate contacted with base with a wash solution.

3. The substrate comprising anionic and cationic ligands of claim 1, wherein the substrate comprises methacrylate, polystyrene, agarose, divinylbenzene, polyacrylamide, cellulose, dextran, or mixtures thereof.

4. The substrate comprising anionic and cationic ligands of claim 1, wherein the base is sodium hydroxide, lithium hydroxide, calcium hydroxide, strontium hydroxide, barium hydroxide, potassium hydroxide, cesium hydroxide, magnesium hydroxide, tetramethylammonium hydroxide, guanidine, rubidium hydroxide, or any combination thereof.

5. The substrate comprising anionic and cationic ligands of claim 1, wherein the base is at a concentration of about 0.1N to about 10N.

6. The substrate comprising anionic and cationic ligands of claim 5, wherein the base is at a concentration of about 4.5N.

7. The substrate comprising anionic and cationic ligands of claim 1, wherein step a) occurs at a temperature of about 15° C. to about 150° C.

8. The substrate comprising anionic and cationic ligands of claim 7, wherein step a) occurs at a temperature of 40° C.

9. The substrate comprising anionic and cationic ligands of claim 1, wherein step a) occurs for about 1 second to about 3 months.

10. The substrate comprising anionic and cationic ligands of claim 9, wherein step a) occurs for about 1 second to about 48 hours.

11. The substrate comprising anionic and cationic ligands of claim 1, wherein a portion of the sulfate, quaternary amine and/or the tertiary amine on the substrate is converted to an anionic group by treating the substrate with the solution comprising a base.

12. The substrate comprising anionic and cationic ligands of claim 11, wherein the anionic group is a carboxymethyl group.

13. The substrate comprising anionic and cationic ligands of claim 11, wherein the substrate comprising anionic and cationic ligands has a ratio of cationic groups to anionic groups that is less than or equal to about 50:1, about 25:1, about 15.5:1, about 2.4:1, about 1.2:1, about 1:1, about 0.7:1, about 0.5:1, about 0.3:1, or about 0.2:1.

14. The substrate comprising anionic and cationic ligands of claim 11, wherein the substrate comprising anionic and cationic ligands has:
a) an amount of anionic groups (ligands) on the surface, relative to the total ionic capacity of the substrate, that are about 5% to about to about 95%, about 7% to about 63%, about 10% to about 43%, or about 19% of the total ionic capacity of the substrate, the total ionic capacity being all (100%) of the anionic groups and cationic groups present on said substrate comprising anionic groups and cationic groups; or
b) the amount of anionic groups (ligands) attached to the surface of the substrate is about X %, where X is any integer between 1 and 99 and the amount of cationic groups (ligands) attached to the surface of the substrate is about Y %, where Y is any integer between 1 and 99, the sum total of the selected X and Y values being 100 (100%).

15. A chromatography column comprising a plurality of substrates comprising anionic and cationic ligands according to claim 1.

16. A method of performing chromatography, the method comprising:
contacting a sample comprising a target molecule to a chromatography column according to claim 15 under conditions such that the target molecule is captured by the plurality of substrate comprising anionic and cationic ligands;
optionally washing the plurality of substrate comprising anionic and cationic ligands with a solution; and
eluting and collecting the target molecule.

17. The method of claim 16, wherein the solution comprises Bis-Tris.

18. A method of making a substrate comprising anionic and cationic ligands comprising:
a) contacting a solution comprising a base with a substrate comprising a sulfate, quaternary amine, and/or a tertiary amine on a surface of the substrate.

19. The method of claim 18, wherein the substrate comprises methacrylate, polystyrene, agarose, divinylbenzene, polyacrylamide, cellulose, dextran, or mixtures thereof.

20. The method of claim 18, wherein the base is sodium hydroxide, lithium hydroxide, calcium hydroxide, strontium hydroxide, barium hydroxide, potassium hydroxide, cesium hydroxide, magnesium hydroxide, tetramethylammonium hydroxide, guanidine, rubidium hydroxide, or any combination thereof.
